(19) United States
(12) Patent Application Publication
(10) Pub. No.: US 2018/0256768 A1
(43) Pub. Date: Sep. 13, 2018

(54) Scented Cutting Implement, and Methods of Making and Using the Same

(71) Applicant: Acme United Corporation, Fairfield, CT (US)

(72) Inventor: Sarah A. Albano, Fairfield, CT (US)

(73) Assignee: Acme United Corporation, Fairfield, CT (US)

(21) Appl. No.: 15/848,888

(22) Filed: Dec. 20, 2017

Related U.S. Application Data
(60) Provisional application No. 62/469,590, filed on Mar. 10, 2017.

(51) Int. Cl. A61L 9/04; B26B 13/22; C08J 3/22

(52) U.S. Cl. CPC A61L 9/042; C08J 3/226; B26B 13/22

(57) ABSTRACT

A cutting implement is disclosed herein comprising a blade section and a handle section, wherein at least a portion of the handle section comprises a scented polymeric composition. Methods of making and using the cutting implement also are disclosed.

### CROSS-REFERENCE TO RELATED APPLICATION

This application claims priority from U.S. Provisional Patent Application No. 62/469,590 filed Mar. 10, 2017.

### BACKGROUND

This disclosure relates generally to cutting implements, including scissors.

Methods are generally known for imparting a long-lasting scent to thermoplastic materials. WO 98/30621 discloses scented plastics in which a scenting agent is incorporated into a vinyl acetate-containing carrier substance, which is then incorporated into a plastic such as polyethylene.

### SUMMARY

One embodiment described herein is a cutting implement comprising a blade section and a handle section, wherein at least a portion of the handle section comprises a scented polymeric composition.

Another embodiment described herein is a method of making a scented cutting implement, comprising obtaining a cutting implement comprising a blade portion and a handle support portion, obtaining a scented polymeric composition, applying the scented polymeric composition to the handle support portion to form a scented polymeric handle, and solidifying the scented polymeric composition.

Yet another embodiment is method of making a scented cutting implement, comprising obtaining a cutting implement comprising a blade portion and a handle support portion, obtaining a first polymeric composition, applying the first polymeric composition to the handle support portion to formed a polymeric handle, obtaining a scented polymeric composition, applying the scented polymeric composition over at least a portion of the first polymeric composition, solidifying the first polymeric composition, and solidifying the scented polymeric composition.

### BRIEF DESCRIPTION OF THE DRAWINGS

FIG. 1 is a perspective view of a first embodiment of scented scissors in a closed position;

FIG. 2 is a front view of the scissors of FIG. 1 in a closed position;

FIG. 3 is a rear diagrammatic view of the scissors of FIG. 1;

FIG. 4 is a diagrammatic top plan view of the scissors of FIG. 1;

FIG. 5 is a bottom plan view of the scissors of FIG. 1;

FIG. 6 is an enlarged left side view of the handle of the scissors of FIG. 1;

FIG. 7 is an enlarged right side view of the handle of the scissors of FIG. 1;

FIG. 8 is a perspective view of the scissors of FIG. 1 illustrated in an opened position;

FIG. 9 is a rear diagrammatic view of the opened scissors of FIG. 8;

FIG. 10 is a sectional view of a second embodiment in which the handles are formed from two layers with a scented outer layer;

FIG. 11 is a front view of a third embodiment of scented scissors;

FIG. 12 is a front view of a fourth embodiment in which a scented layer is selectively applied over a first layer of the handles; and

FIG. 13 is a front view of a knife having a scented handle.

### DETAILED DESCRIPTION

The embodiments described herein include cutting implements with scented polymeric handles and method of making and using the cutting implements. In some cases, the cutting implement is children's scissors that are imparted with an appealing scent, such as a fruit scent or peppermint. In certain cases, the cutting implement is adult scissors or a knife.

As used herein, the term “scented polymeric composition” means a polymeric composition that has a pleasing scenting agent incorporated therein such that the scented polymeric composition emits an odor that can be detected by an average human nose from a distance of at least about eight inches or less. The polymeric composition can be in the form of a solid. In embodiments, the scenting agent is supported by a carrier.

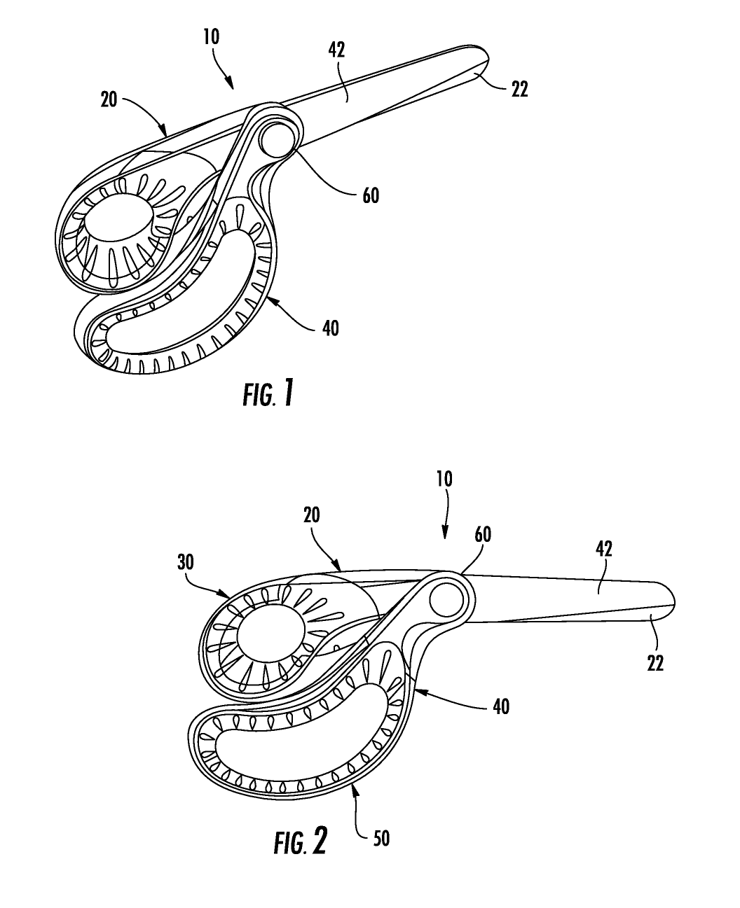

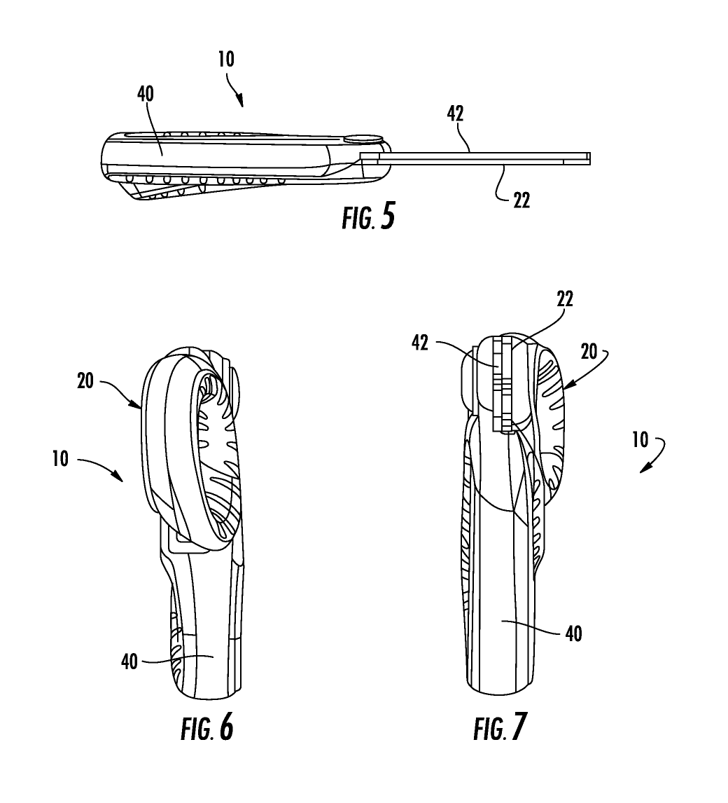

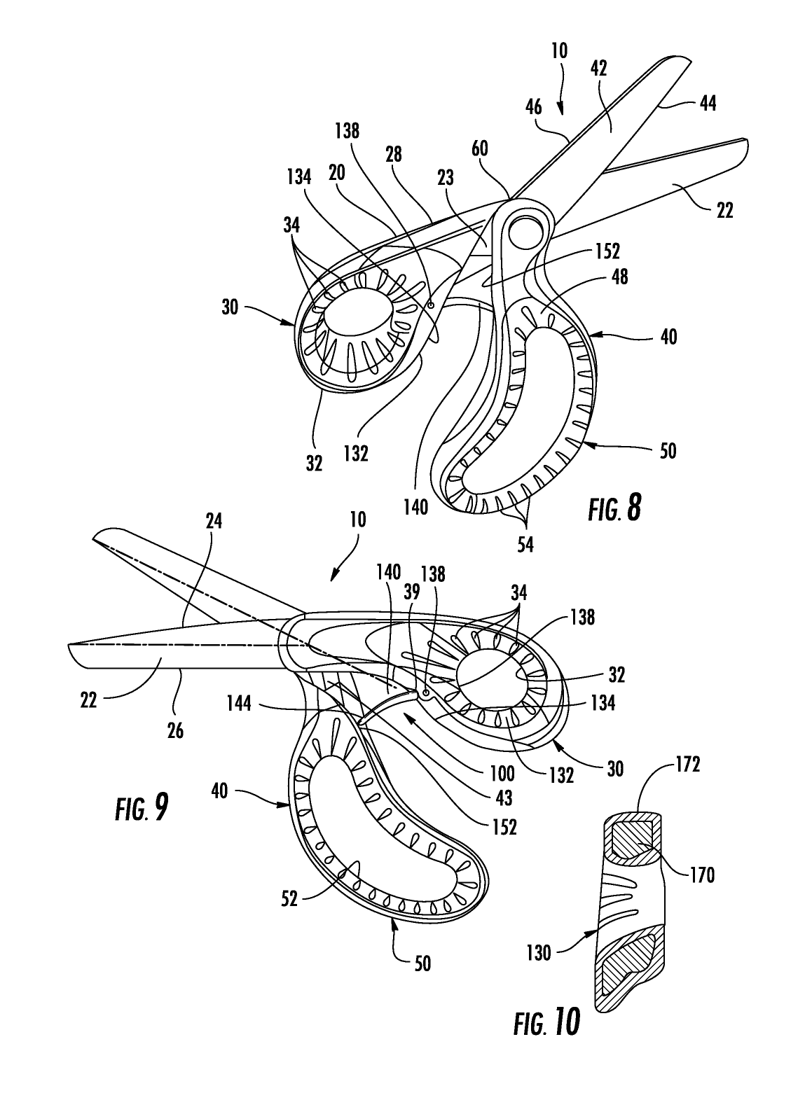

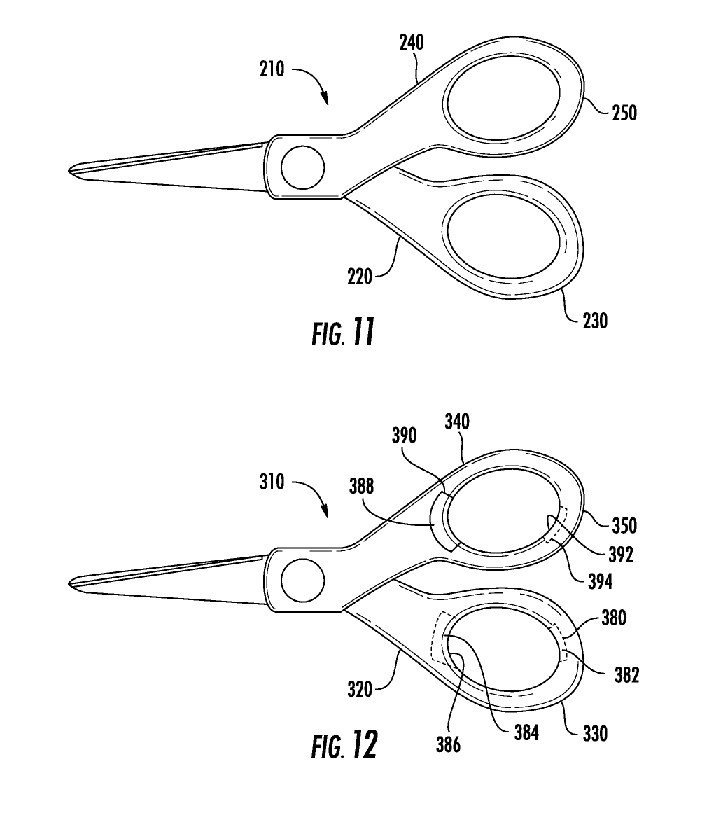

With reference to FIGS. 1-9 wherein like numerals represent like parts throughout the figures, a pair of scented scissors is generally designated by the numeral 10. Scissors 10 comprises a first scissor 20 and a second scissor 40 which are pivotally joined at a pivot connecting assembly 60.

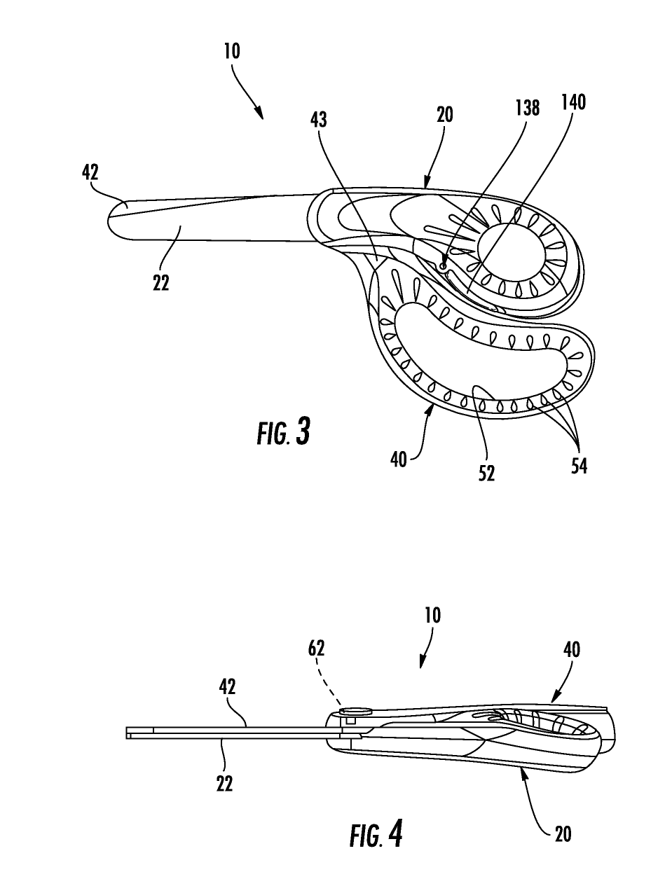

First scissor 20 includes a blade 22 that is most cases is somewhat dull at its cutting edge 24 and contoured or beveled at the opposite edge 26. A shank 23 integrally extends from the blade. The blade 22 and the shank 23 are generally co-linear or only slightly angled to each other. An overmold 28 integrally extends rearwardly over the shank 23 (partially illustrated) to form a rear handle 30. The handle 30 defines a thumb opening 32. The thumb opening 32 is dimensioned to accommodate a thumb of a child. The representative dimensions in FIGS. 3-5 are in millimeters.

The handle 30 is preferably formed of a soft molded thermoplastic material (TP), such as polypropylene (PPE), a thermoplastic elastomer (TPE), or a natural or synthetic thermoset material (TS). Non-limiting examples of suitable materials are described below in further detail. In embodiments, the handle 30 has a smooth two-color contour which tapers very gradually from a central portion toward the thumb opening, however, a single color alternatively can be used. The first handle 30 has an inner exterior surface 132. Optionally, numerous shallow indentations 34 extend from proximate the opening 32 of both sides of the first scissor onto the handle to form an interrupted surface to facilitate an effective grip for the child. The medial portion of the first scissor 20 includes an opening (not illustrated) for receiving a pivot connecting assembly 60, as will be later described.

The second scissor 40 has a crank-like configuration which forms a forward blade 42 which has a generally dull cutting edge 44 and an opposite contoured or beveled edge 46. The cutting edge 44 cooperates with cutting edge 24 to implement the cutting function. The second scissor 40 has a medial opening (not illustrated) which receives the pivot assembly 60.

A rear angled shank 43 (partially illustrated) integrally extends rearwardly from the blade 42 in an integral fashion and is preferably bifurcated. In embodiments, the offset angle A between the blade 42 and the shank 43 is preferably approximately 30° and may have an angle in the range of approximately 15°-45° to provide the desired ergonomic function. An overmold 48 of soft material, such as thermoplastic, thermoset, or thermoplastic elastomer, covers the shank end portion to form a rear handle 50 of the second scissor. The handle 50 defines an oblong arcuate finger opening 52 for accommodating the fingers of a child. Numerous shallow indentations 54 extend from proximate the finger opening 52 onto the outer surface of the overmold 48 on both sides of the handle to form an effective grip surface. The handle 50 preferably has a soft, smooth two-color configuration.

Suitable non-limiting examples of non-toxic thermoplastic or thermoset material for the handle include polypropylene (PP), polyethylene (PE), polyvinyl chloride (PVC) a thermoplastic elastomer such as ethylene-vinyl acetate (EVA) which is a copolymer of ethylene and vinyl acetate, or a thermoset material such as natural or synthetic rubber. In embodiments, the handle is formed from a foamed polymeric material.

The scent composition can be directly combined with the polymeric handle material, and/or can be applied in a coating over a portion or all of the polymeric handle material. In embodiments, the scent is incorporated into a carrier, such as an ethylene-vinyl acetate copolymer, which is combined with the thermoplastic or thermoset material for the handle. In embodiments, the scented polymeric composition is encapsulated by an encapsulating agent.

The location of the scenting agent can be throughout the handle material, incorporated into an outer layer on the handle material, or incorporated only into certain sections of the handle material. In embodiments, the scissors include a thumb opening and a finger opening, and the scented polymeric composition is disposed within at least one of the thumb opening and the finger opening. This configuration is useful when scented polymeric composition is configured to release a scent upon application of pressure from at least one of a finger and a thumb.

In some cases, if the handle of the cutting implement includes ergonomic indentations, such as are shown in FIGS. 1-10 above, the scented polymeric composition is applied inside the indentations as an outer coating layer. The scented polymeric composition may partially fill the indentations, completely fill the indentations, or extend outwardly from the surrounding outer surface of the handle section. In embodiments, the scented polymeric composition optional may have a higher or lower Shore A hardness, and/or and higher or lower coefficient of friction, as compared to the surrounding portions of the handle section.

With reference to FIG. 4, the pivot assembly 60, which may include a fastener or pin 62 (shown in phantom) of various forms, connects through the openings (not illustrated) of the first scissor 20 and the second scissor 40, respectively, and is secured by various forms to pivotally join the scissors. A light torsion spring (not illustrated) may be received in cooperative recesses surrounding the openings, respectively. The torsion spring preferably surrounds the central fastener pin 62. The spring includes ends which are anchored at openings or anchors, respectively, in opposed adjacent sides of the first and second scissors proximate the pivot. The optional spring functions to assist the child by providing a small spring force to open the scissors to facilitate cutting. Alternative or no spring configurations may be provided.

Optionally, the scissors include a leaf member 140 to establish a minimum acute angle opening angle for the scissors. In embodiments, the rear portion of the handle 30 surface includes a limit, or stop 39 which limits the pivoting of the leaf member 140. When the leaf member is extended, a pre-established blade opening is formed that allows for a child to easily open and close the scissors to make cuts that are short in length. It will be appreciated that the leaf member 140 is pivotally positionable around a pin 138 from a retracted position totally captured in the recess to an extended position wherein the distal end 144 of the leaf member engages a surface 152 of the handle 50. In some cases, the stop 39 is formed from the same material as the handle 30. It will be appreciated that when the scissors are not used and are placed in the blade closed position, such as illustrated in FIGS. 1-3, the leaf member 140 is rearwardly pivoted into the handle recess 134. For more advanced usage by a child, the leaf member 140 can be maintained in the retracted position, and the child can operate the scissors in a more normal fashion.

FIG. 10 shows a cross sectional view of an embodiment of a scissor handle 130 that include an inner polymeric layer 170 and a scented outer polymeric layer 172 formed over the inner polymeric layer 170. In this embodiment, all sides of the handle 130 are coated with the scented outer polymeric layer 172.

FIG. 11 shows adult scissors 210 with a first scissor 220 having first scented handle 230 and second scissor 240 with a second scented handle 250. The handles are of substantially the same size as one another.

FIG. 12 shows adult scissors 310 with a first scissor 320 having a first handle 330 and second scissor 340 with a second handle 350. The first handle 330 has a first finger contact portion 380 with a first scented coating layer 382 formed thereon and, on the opposite side (shown in broken lines as it is on the underside of the scissors), a second finger contact 384 with a second scented coating layer 386 formed thereon. The second handle 350 has a third finger contact portion 388 with a third scented coating layer 390 formed thereon, and, on the opposite side (shown in broken lines as it is on the underside of the scissors) a fourth scented coating layer 392 with a fourth scented coating layer 394 formed thereon. The scented coating layers 382, 386, 390 and 394 are formed from a polymeric material that releases scent upon application of pressure during use of the scissors.

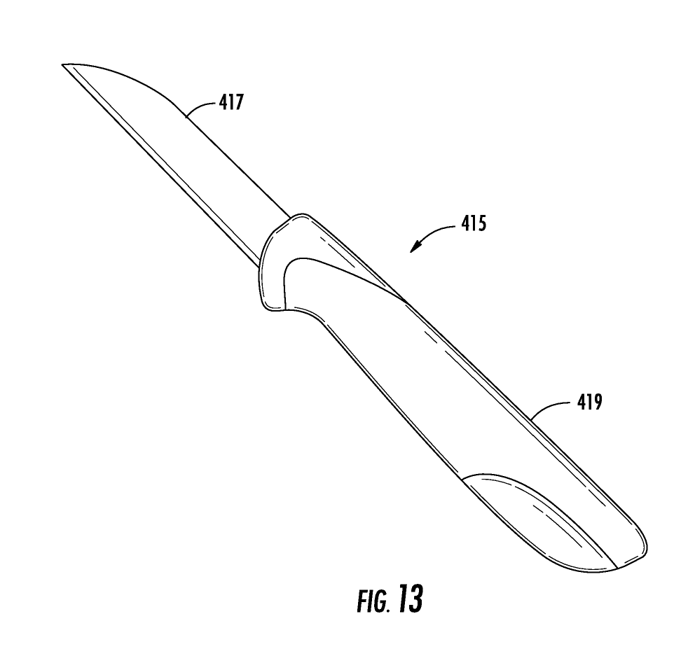

FIG. 13 shows a knife 415 having a blade 417 and a scented polymeric handle 419. In some cases, the entire handle is formed from the scented polymeric material. In other cases, a first part of the handle is formed from the scented polymeric material and a second part of the handle is formed from an unscented polymeric material.

In some cases, the scent composition is oil based, i.e. not water soluble. In embodiments the scent composition comprises at least one of a fruit scent, a mint scent, and a floral scent. Non-limiting examples of scent compositions, including the carrier, are provided below. For the fruit flavored scents, if the vanillin content is zero, the fruit fragrance composition is at least 0.05 wt %. These scented polymeric compositions are incorporated into a primary handle material in an amount of about 0.5 to about 15 parts by weight of scenting agent based upon 100 total parts by weight of the total handle composition, or about 1 to about 10 parts by weight of scenting agent, or about 3 to about 8 parts by weight of scenting agent.

Fruit Flavored Scents

| | | |
|---|---|---|
| Vanillin (natural or synthetic) | 0-12 | wt % |
| Ethyl maltol | 0-2 | wt % |
| Ethyl butyrates | 0-3 | wt % |
| Ethyl vinyl alcohol | 70-95 | wt % |
| Fruit fragrance and | 0-20 | wt % |
| other additives | | |

Peppermint Scents

| | | |
|---|---|---|
| Peppermint oil | 5-20 | wt % |
| Ethyl vinyl alcohol | 80-95 | wt % |
| Other compounds | 0-10 | wt % |

Floral Scents

| | | |
|---|---|---|
| Floral Fragrance | 0.05-12 | wt % |
| Ethyl maltol | 0-2 | wt % |
| Ethyl butyrates | 0-3 | wt % |
| Ethyl vinyl alcohol | 70-95 | wt % |

Non-limiting examples of suitable fragrances to impart the scent include leaf extracts, strawberry acid, blueberry extract, black currant extract, orange extract, apple extract, orange oil, grapefruit extract, etc. The scent can be supplied in powder form or in the form of a liquid. Non-limiting examples of plant and floral fragrances include rose, orange blossom, lavender, honeysuckle, jasmine, pine and gardenia extracts. When the scent is supplied as a powder, it can be homogeneously blended with a base material and other additives. For best results, a blender that ensures thorough mixing is used.

A non-limiting example of a scented polymeric composition that can be used to form the handle section of the cutting implement contains about 20-30 parts by weight of polyvinyl chloride, 5-8 parts by weight of nitrated rubber, 4-8 parts by weight of calcium carbonate, 6-10 parts by weight of calcium stearate, 2-4 parts by weight of thickener, and 1-10 parts by weight of plant flavorings. Non-toxic powders also may be incorporated into the formulation, typically in an amount of about 3 to about 20 parts by weight. All parts by weight are based upon 100 parts by weight of the total composition. A foaming agent also can be used, typically in an amount of about 3-5 parts by weight based on a total composition of 100 parts by weight. Non-toxic powders can be added in amounts of about 4 to about 20 parts.

In embodiments, the scent is incorporated into the polymeric composition by blending and stirring, and typically is added after the other materials have been melted and thoroughly mixed.

In another embodiment, the application of pressure releases scent particles into the air. In such cases, the fragrance particulates can be encapsulated in capsules that are configured to be ruptured when manual pressure is applied during use of the cutting tool in order to release fragrance particles. In embodiments, the capsules are configured in order that fragrance particles are not released when pressure is not being applied to the capsules. This configuration may provide for long term release of scent from the cutting tool.

While preferred embodiments of the foregoing have been set forth for purposes of illustrating the children's ergonomic scissors, the foregoing description should not be deemed a limitation of the invention herein. Accordingly, various modifications, adaptations and alternatives may occur to one skilled in the art without departing from the spirit and the scope of the present invention.

A number of alternatives, modifications, variations or improvements therein may be subsequently made by those skilled in the art, which are also intended to be encompassed by the following claims.

What is claimed is:

1. A cutting implement comprising a blade section and a handle section, wherein at least a portion of the handle section comprises a scented polymeric composition.

2. The cutting implement of claim 1, wherein the handle section has a generally uniform composition.

3. The cutting implement of claim 1, wherein the handle section has at least first and second layers, and the second layer comprises the scented polymeric composition.

4. The cutting implement of claim 1, wherein the scented polymeric composition comprises a coating layer formed over only a portion of the handle section.

5. The cutting implement of claim 1, wherein the cutting implement is scissors.

6. The cutting implement of claim 1, wherein the cutting implement is children's scissors.

7. The cutting implement of claim 5, wherein the scissors include a thumb opening and a finger opening, and the scented polymeric composition is disposed within at least one of the thumb opening and the finger opening.

8. The cutting implement of claim 7, wherein the scented polymeric composition is configured to release a scent upon application of pressure from at least one of a finger and a thumb.

9. The cutting implement of claim 1, wherein the cutting implement is a knife.

10. The cutting implement of claim 1, wherein the cutting implement is a sewing tool.

11. A method of making a scented cutting implement, comprising:
obtaining a cutting implement comprising a blade portion and a handle support portion,
obtaining a scented polymeric composition,
applying the scented polymeric composition to the handle support portion to form a scented polymeric handle, and
solidifying the scented polymeric composition.

12. The method of claim 11 wherein the scented polymeric composition comprises a thermoset material.

13. The method of claim 11, wherein the scented polymeric composition comprises a thermoplastic material.

14. The method of claim 11, wherein the scented polymeric composition comprises a thermoplastic elastomer.

15. The method of claim 11, wherein the scented polymeric composition comprises a liquid.

16. The method of claim 11, wherein a scent composition is incorporated into microcapsules in the scented polymeric composition.

17. A method of making a scented cutting implement, comprising:
obtaining a cutting implement comprising a blade portion and a handle support portion,
obtaining a first polymeric composition,
applying the first polymeric composition to the handle support portion to formed a polymeric handle,
obtaining a scented polymeric composition,
applying the scented polymeric composition over at least a portion of the first polymeric composition,
solidifying the first polymeric composition, and
solidifying the scented polymeric composition.

18. The method of claim 17, wherein the first polymeric composition is solidified before application of the scented polymeric composition.
